(19) United States
(12) Patent Application Publication
(10) Pub. No.: US 2024/0425844 A1
(43) Pub. Date: Dec. 26, 2024

(54) SALT PLUG INJECTION METHODS FOR IMPROVED ANION EXCHANGE ANALYSES OF LARGE NUCLEIC ACIDS

(71) Applicant: Waters Technologies Corporation, Milford, MA (US)

(72) Inventors: Szabolcs Fekete, Ville La Grand (FR); Matthew Lauber, North Smithfield, RI (US)

(73) Assignee: Waters Technologies Corporation, Milford, MA (US)

(21) Appl. No.: 18/748,243

(22) Filed: Jun. 20, 2024

Related U.S. Application Data
(60) Provisional application No. 63/509,835, filed on Jun. 23, 2023.

(51) Int. Cl. C12N 15/10; C12Q 1/6806

(52) U.S. Cl. CPC C12N 15/101; C12Q 1/6806

(57) ABSTRACT

The present technology is directed to methods of improved nucleic acid-based ion-exchange chromatography. In particular, the methods utilize a modification of a sample, and in some examples a modification of mobile phase carrying the sample to the ion-exchange column. In a method of the technology, a bracketed sample is loaded into an injector needle apparatus and injected into an ion-exchange column. The bracketed sample is loaded by (a) adding a first-plug solvent comprising a first salt and having a basic pH; (b) adding a sample plug; and (c) adding a second-plug solvent comprising a second salt and having a basic pH. In certain examples, the bracketed sample is delivered to the column using a mobile phase gradient consisting of first solvent and second solvent and wherein 0% of the second solvent is excluded from the gradient.

### RELATED APPLICATIONS

This application claims the benefit of and priority to U.S. Provisional Application No. 63/509,835, filed Jun. 23, 2023, the entire disclosure of which is incorporated herein by reference.

### FIELD OF THE TECHNOLOGY

The field of the present technology relates to injection techniques for improved anion exchange analyses of large nucleic acids. In particular, the present technology relates to utilizing one or more salt plug solvents when injecting a sample into an ion-exchange column. The methods of the present technology provide improved nucleic acid-based chromatographic separation.

### BACKGROUND

Anion exchange has significant potential for use as an analytical technique for mRNA concentration and integrity determination, but current methods tend to exhibit high carryover that can make the techniques impractical to implement.

With a burgeoning pipeline of nucleic acid-based therapies, there is a need for improved analytical methods that can quickly confirm the concentration, integrity and relative abundance of various large nucleic acid species.

### SUMMARY

The present technology solves the above need by providing unique and robust ion-exchange chromatography methods for analyzing nucleic acids in samples.

In particular, methods of the present technology advantageously solve a known problem of anion exchange (“AEX”) separations for nucleic acids analytes, which is the lack of method robustness due to severe carryover effects (typically on the order 10-20% of a previous analyte/sample being present in a subsequent separation). Conventional methods have tried to address the carryover issue by using conditioning or blank sample runs. The methods of the present technology are able to prevent/minimize carryover without the need of running several consecutive blank runs and conditioning runs. As a result, the methods of the present technology provide both a savings in cost and time with the elimination of the costly and time-consuming conditioning and sample blank runs.

In an aspect, the present technology is directed to a method of ion-exchange chromatography comprising the steps of: (a) loading a bracketed sample into an injector needle apparatus by: adding a first-plug solvent; adding a sample plug; and adding a second-plug solvent; and (b) injecting the bracketed sample into an ion-exchange column for chromatographic separation. The first-plug solvent comprises a first salt and has a basic pH (e.g., a pH of about 10). The second-plug solvent comprises a second salt and also has a basic pH (e.g., a pH of about 10). Preferably, the first-plug solvent and the second-plug solvent are the same solvents (e.g., identical or comprise the same composition). However, they need not be.

The above aspect can include one or more of the following features. In some examples, the method features an additional step of injecting into the ion-exchange column a subsequent salt plug injection subsequent to injecting the bracketed sample. In some examples, the subsequent salt plug injection is greater in volume than the first-plug solvent or the second-plug solvent. Some examples feature an ion-exchange column that contains a weak anion exchanger comprising a diethylaminoethyl-functionalized non-porous resin. In some examples, the sample comprises a nucleotide, oligonucleotide, nucleic acid, or fragments or derivatives thereof. In some examples, the first salt of the first-plug solvent and/or the second salt of the second-plug solvent comprises sodium bromide or guanidine HCl. In certain examples, the first salt concentration of the first-plug solvent and/or the second salt concentration of the second-plug solvent is greater than 1M of sodium bromide (e.g., 2M of sodium bromide). In some examples, the volumetric ratio of first-plug solvent to second-plug solvent that is in the injector needle apparatus is 1:1. In certain examples, the each of the first-plug solvent and the second-plug solvent further comprises a buffer. In some examples, a solvent plug volume (i.e., the volume of the first-plug solvent and the second-plug solvent) is at least equal to a sample volume. In certain examples, the solvent plug volume is about 1.2 times the sample volume. In some examples, the bracketed sample is injected into the ion-exchange column using a mobile phase gradient. The mobile phase gradient comprises using a first solvent and a second solvent and excludes the use of 0% of the second solvent (i.e., gradient does not include starting at 0% of the second solvent, but at a higher amount, such as 1%). In some examples, the ion-exchange column is run at or above ambient temperature (e.g., 27 C, 35 C).

In another aspect, the present technology is directed to a method of ion-exchange chromatography comprising the steps of loading a sample into an injector needle apparatus to form a modified sample by: adding a salt plug solvent comprising 2M NaBr and a buffer; adding the sample; and injecting the modified sample into an ion-exchange column for chromatographic separation. The salt plug solvent has a basic pH (e.g., a pH of about 10).

The above aspect can include one or more of the following features. In some examples, the salt plug solvent is added prior to adding the sample. In other examples, the salt plug solvent is added after adding the sample. In some examples, the method features an additional step of injecting into the ion-exchange column a subsequent salt plug injection subsequent to injecting the modified sample. In some examples, the subsequent salt plug injection is greater in volume than the salt plug solvent. Some examples feature an ion-exchange column that contains a weak anion exchanger comprising a diethylaminocthyl-functionalized non-porous resin. In some examples, the sample comprises a nucleotide, oligonucleotide, nucleic acid, or fragments or derivatives thereof. In some examples, a ratio of salt plug volume to sample volume ranges from about 1 to about 1.2. In some examples, the modified sample is injected into the ion-exchange column using a mobile phase gradient. The mobile phase gradient comprises using a first solvent and a second solvent and excludes the use of 0% of the second solvent (i.e., gradient does not include starting at 0% of the second solvent, but at a higher amount, such as 1%). In some examples, the ion-exchange column is run at or above ambient temperature (e.g., 27 C, 35 C).

### BRIEF DESCRIPTION OF DRAWINGS

The technology will be more fully understood from the following detailed description taken in conjunction with the accompanying drawings, in which:

FIG. 1 shows an illustration of macromolecules that adsorb to a surface (i.e., stationary phase) based on a spreading phenomenon caused by strong ionic charges of a nucleic acid analyte.

FIG. 2A shows a schematic example of the present technology using a bracketed injection approach.

FIG. 2B shows a schematic example of a typical, unbracketed sample injection.

FIG. 3A shows the effect of an mRNA solute's binding (residence) time on mRNA carryover in an anion exchange separation (AEX).

FIG. 3B shows the initial mobile phase strength on mRNA carryover in AEX.

FIG. 4 shows the effect of salt plug solvent volume to sample volume ratio (V_{mod}/Vₛₐₘₚₗₑ) on carryover (solid line curve) and recovery (dotted line curve).

FIG. 5A, FIG. 5B, and FIG. 5C show carryover for various samples observed for Gen-Pak FAX (a diethylaminoethyl-functionalized non-porous resin commercially available from Waters Technologies).

FIG. 5D, FIG. 5E, and FIG. 5F show carryover for various samples observed for Monolithic WAX columns.

FIG. 6A, FIG. 6B, FIG. 6C, FIG. 6D, FIG. 6E, and FIG. 6F show the effect of temperature on the chromatographic profile (selectivity) and carryover.

FIG. 7 shows the time of a subsequent salt plug injection after the initial injection of a bracketed injection.

FIG. 8A, FIG. 8B, and FIG. 8C show bracketed injection carryover for different mRNA samples.

FIG. 8D, FIG. 8E, and FIG. 8F show carryover after a subsequent salt injection made after the bracketed injection for different mRNA samples.

FIG. 9 shows the carryover effect based on the volumetric solvent/sample plug ratio across various salt plugs with that vary in pH (9.5-11.5).

FIG. 10A shows the effect of solvent salt plugs of pH of 10.2 for EPO mRNA.

FIG. 10B shows the effect of solvent salt plugs of pH of 11.5 for EPO mRNA.

### DETAILED DESCRIPTION

### Definitions

As used herein, the term “approximately” or “about” means+/−10% of the recited value.

As used herein, the term “includes” means includes but is not limited to, and the term “including” means including but not limited to.

As used herein, the term “eluent” refers to a carrier portion of the mobile phase, such as a solvent or mixture of solvents with which a sample can be delivered in a chromatographic process.

As used herein, the term “eluate” refers to the material that emerges from or is eluted from a chromatographic process. To “elute” a molecule with an “eluent solution” (e.g., an oligonucleotide of interest or an impurity) from sorbent is meant to remove the molecule therefrom by altering the solution conditions such that buffer competes with the molecule of interest for binding to the sorbent. A non-limiting example is to elute a molecule from a sorbent by altering the pH of the buffer surrounding the sorbent.

As used herein the term “nucleotide” or “oligonucleotide” refers to a polymer sequence of two more nucleic acids, including RNA, DNA, their analogs, including those having base modifications, sugar modifications or linkers used to modify the bioavailability. Nucleotides and oligonucleotides broadly include, but are not limited to, nucleotides from double-stranded RNA, single-stranded RNA, single stranded DNA, double stranded DNA, double standard RNA/DNA hybrid, synthetic RNA, synthetic DNA, and combinations thereof.

As used herein, the term “stationary phase” refers to the phase or portion that is fixed in place or stationary in a chromatographic process, such as a solid material within a column through which the mobile phase passes.

As used herein, the term “mobile phase” refers to a phase or portion that moves in a chromatographic method, such as by passing through a column, and it includes the sample and the eluent.

As used herein, the term “functionalized,” “modified” or “chemically modified” refers to a changed state or structure of a molecule of this technology. Molecules may be modified in many ways including chemically, structurally, and functionally.

As used herein, the terms “extracting,” “separating,” or “isolating,” as used interchangeably herein, refer to increasing the degree of purity of a target molecule, i.e., one or more analytes, a solution comprising the target molecule and one or more impurities. Typically, the degree of purity of the target molecule is increased by removing (completely or partially) an impurity from a composition.

As used herein, and unless stated otherwise, the term “sample” refers to any composition or mixture that contains one or more analytes of interest. In an example, the sample is a nucleotide, oligonucleotide, nucleic acid, or fragments or derivatives thereof. Samples may be derived from biological or other sources. Biological sources include eukaryotic and prokaryotic sources, such as plant and animal cells, tissues and organs. The sample may be “partially purified” (i.e., having been subjected to one or more purification steps, such as filtration steps) or may be obtained directly from a host cell or organism producing the one or more analytes of interest (e.g., the sample may comprise harvested cell culture fluid).

As used herein, the term “carryover” refers to the presence of a peak or response from a previous chromatographic run appearing in subsequent runs.

The present technology addresses the need for isolating and analyzing biological substances from samples using chromatography and, in particular, ion-exchange chromatography without interfering carryover. In conventional methods, carryover can range from 5-20% of an amount of analyte of a sample that is observed in a blank injection following the sample injection.

In particular, the present technology addresses the above need by employing methods of ion-exchange chromatography that involve loading an injector needle apparatus with one or more solvent plugs that are adjacent to or bracket a sample. The sample and solvent plug(s) are then injected into an ion-exchange chromatography column.

In an example, the present technology is directed to a method of ion-exchange chromatography comprising the steps of: (a) loading a bracketed sample into an injector needle apparatus by: adding a first-plug solvent; adding a sample plug; and adding a second-plug solvent; and (b) injecting the bracketed sample into an ion-exchange column for chromatographic separation. The first-plug solvent comprises a first salt and has a basic pH (e.g., a pH of about 10). The second-plug solvent comprises a second salt and also has a basic pH (e.g., a pH of about 10). Preferably, the first-plug solvent and the second-plug solvent are the same solvents (e.g., identical or comprise the same composition). However, they need not be.

The present technology decreases carryover by shortening the analyte's column residence time by applying an injection approach where the drawn sample is adjacent to at least one salt plug solvent, which may modulate the strength of solute binding at the head of the column. In certain embodiments, the drawn sample is sandwiched between a first salt plug solvent and a second salt plug to provide a bracketed sample. In some embodiments, the present technology can further shorten an analyte's column residence time by use of an appropriately designed gradient of the mobile phase. In particular, column residence time (and thus carryover) can be reduced by starting the mobile gradient with comparatively high counter ion concentrations (i.e., mobile phase B concentration begins at a non-zero concentration, e.g., 2%, 3%, 4%, 5%, etc.).

Without being bound by theory, the present technology addresses problems caused by the interactions of the analyte with an ionic stationary phase. Specifically, the present technology addresses the effective force of interaction between two charges (ionic, F∝1/d²)—the charged solute containing the analyte and the anionic exchanger stationary phase. This type of interaction results in the surface spreading of macromolecules of the sample on the stationary phase.

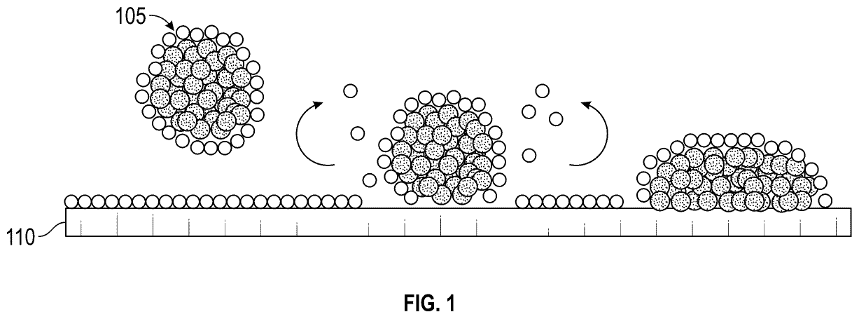

As shown in FIG. 1, if a solute (105) with a strong charge is close enough to a surface (110) of the stationary phase, even without the need of contacting the stationary phase, it will be attracted to the surface, which leads to the problem of surface spreading. The acidic dissociation constant of nucleic acids' phosphate is very low (pKa of about 2.14) while the pKa of anion exchangers are greater than 10.5.

FIG. 1 depicts an illustration of the surface spreading phenomenon. Surface spreading of molecules, including nucleic acids, are caused by strong charges that cause adsorption, which can be irreversible over time and impacts preferred binding orientation and mono and multi-layer adsorption. Surface spreading depends on time, ionic strength, temperature, and pH. The methods of the present technology mitigate the charged interactions in the sample that cause this spreading phenomenon.

Macromolecules, in general, are surface-active molecules, which undergo non-specific adsorption when they come into contact with different types of surfaces. Non-specific adsorption can cause a loss of solute content or result in aggregation. During an adsorption event, most macromolecules undergo shape changes (conformational changes like unfolding). The area on the adsorbent surface that is occupied by the large macromolecules is referred to as “the footprint.” The footprint usually increases with residence time, which can be referred to as a “spreading process.” Footprint-related extra adsorption is usually partially reversible. The adsorption kinetics and footprint surface area strongly depend on the solute concentration as well.

Without being bound by theory, the phenomenon behind the poor injection repeatability and high carryover effects observed with biomolecules (macromolecules) is related to non-desired secondary interactions with surfaces and to both inter- and intramolecular interactions occurring in a macromolecular system.

At high analyte concentrations, a surface comes to be occupied in a shorter time and thus the time available for spreading is then shorter as well. This results in a smaller average footprint; however, the adsorbed concentration will be higher. Other parameters such as solvent pH and ionic strength may also impact the size and spreading of the footprint.

To address the spreading phenomenon, the present technology modifies the sample (prior to injection into an ion-exchange column) to counteract the charges of the analytes. Specifically, and in one example, the present technology injects the sample with a salt plug solvent adjacent to the sample. Without being bound by theory, the present technology addresses the issue that mRNAs are bound at the head of the column and remain motionless until they experience the eluting mobile phase composition of a gradient. Hence, a certain time is available for analyte spreading to occur and for multipoint interactions to form with the stationary phase. A shorter residence time leads to fewer binding segments and in turn weaker adsorptive interactions and lower carryover.

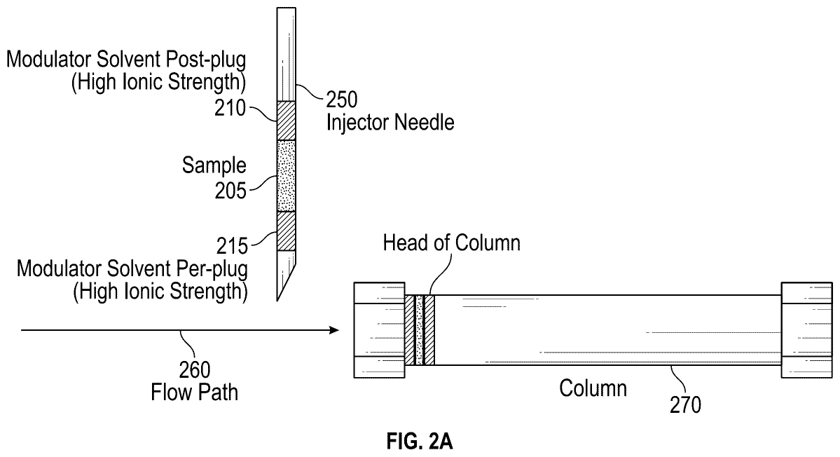

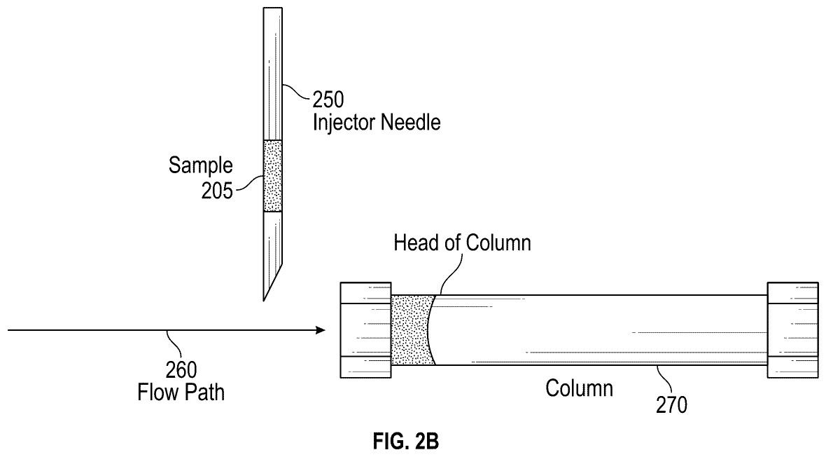

To illustrate this, FIG. 2A presents an example of the present technology in the form of a bracketed injection. In this example, a sample (205) is modified by the addition of a first-plug solvent (210) located upstream and adjacent to the sample (205), and by the addition of a second-plug solvent (215) located downstream and adjacent to the sample (205). The first-plug solvent (210) and the second-plug solvent (215) together with the sample form a bracketed sample within an injection needle (250). The first-plug solvent (210) and the second-plug solvent (215) modify the interaction of the sample (205) with the stationary phase within column (270) by providing a high concentration of counterion. That is, the first-plug solvent (210) and second-plug solvent (215) have a basic pH, approximately a pH of 10 or more. The bracketed sample is injected into flow path (260) containing the mobile phase and is delivered to the column (270) (i.e., injected into the column). Even though analytes, such as oligonucleotides in the sample (205) have a pKa value much lower than that of the stationary phase within the ion-exchange column (270), the first-plug solvent (210) and the second-plug solvent (215) (each having a basic pH) reduce the interaction between the sample (205) and the stationary phase to reduce or eliminate spreading. That is, when the bracketed sample is injected into the (e.g., AEX) column (270) and forms an analytical gradient, an unexpected substantial decrease in carryover is observed (see, FIG. 2A-FIG. 2B). In general, using the bracketed sample of this exemplary method reduced carryover to about 2%, which is far less than 5-20% carryover observed without this method.

The present technology is not limited to bracketing. In an alternative example, the method involves modifying the sample by the addition of a single plug solvent adjacent to the sample plug in the injection needle either upstream or downstream from the sample. In some examples, the plug solvent is added to prior to adding the sample. In some examples, the plug solvent is added after adding the sample.

In some examples, samples or salt plugs may be added to the injection needle by aspirating or via a flow path.

The aforementioned plug solvents of the present technology preferably comprise one or more salts with a high counterion concentration. Salts used as counterions are not particularly limited. In some examples, salts one or more of sodium bromide, guanidine hydrochloride, NaCl, and (NH₄)₂SO₄ among others.

For AEX of nucleic acid-based analytes such as mRNAs, where the binding interaction is inherently very strong, the present technology uses solvent plugs that bracket the sample or are adjacent to the sample and have a strong enough salt concentration to limit the strength of the initial binding. While the amount of salt is not particularly limited, the salt concentration should be high enough to reduce the interaction between the sample and the stationary phase to reduce or eliminate spreading. Therefore, the solvent plugs contain high concentrations of salt (counterion). In some examples the salt concentration is greater than 100 mM, greater than 1M, or greater than 2M. In a preferred example, the concentration of salt is 2M.

In an example, the plug solvents contain a buffer. Buffers are not particularly limited so long as the buffering agent is biocompatible and does not denature the nucleic acid or other analyte of interest. In a preferred example, the buffer is a TRIS buffer. A pH concentrate that is diluted with water to adjust the pH such as by using BioResolve™ CX pH concentrates A or B commercially available from Waters Technologies may also be used as a buffer.

In an example, the total volume of solvent plug to sample volume ratio is about 1-1.2. One of ordinary skill in the art recognizes that the optimal (V_{mod}/Vₛₐₘₚₗₑ) ratio may depend on the sample, injector apparatus, system volumes, mobile phase and column. It needs to be optimized individually for each method setup. As an example, if 2 μL of mRNA sample is going to be injected, then one of ordinary skill in the art may program a sequence with a 1 μL salt solvent plug+2 μL sample+1 μL salt solvent plug. In a preferred example, the ratio is about 1.

The stationary phase materials used in the methods of the present technology are not particularly limited so long as it is capable of separating the analytes of interest of the samples. In a preferred example, the stationary phase comprises functional groups used in a weak anion exchanger. In some examples, the weak anion exchanger (“WAX”) is in the form of a monolith. In a preferred example, the AEX is a weak anion exchange comprising a diethylaminocthyl-functionalized non-porous resin such as in Gen-Pak FAX columns commercially available from Waters Technologies Corporation, Milford, MA.

While the pH of the plug solvents is not particularly limited, the pH is optimally at a basic pH such as by using, e.g., a buffering agent. In a preferred example, the pH is about 10. Although higher pH improves reduction of carryover, the pH cannot be too high (e.g., at pH higher than about 11) since mRNAs may be denatured, and the base-pairing and base-stacking interactions might be disrupted. At too high pH, the intact RNA can thereby be linearized which can lead to spreading, a larger binding footprint, and significantly stronger adsorption to the stationary phase material. Furthermore, some nucleobases can be deprotonated above pH 10, which would add additional negative charge to the nucleic acid analyte. This is probably the reason why at high pH condition, the retention of mRNAs increases in AEX. Therefore, a too high pH (>11) is not beneficial for use as a solvent plug. In addition to high ionic strength, the pH of the solvent plug can also be adjusted to be close to the pKa of the stationary phase functional groups (i.e., pH 10-11 in the case of a weak anion exchanger). The combined effect is intended to limit the strength of the initial adsorption.

The ratio of modulating salt plug volume to sample plug volume may be optimized through routine experimentation based on the lowest carryover while avoiding sample breakthrough.

In some examples, the above methods may further comprise a subsequent salt plug injection. Further reductions in carryover can be obtained by adding a salt plug injection at the end of the analytical gradient (i.e., a subsequent plug-solvent). For example, carryover in a mRNA analysis was reduced to about 1% when a salt plug injection was added after the bracketed sample was injected and isolated. The subsequent plug-solvent can be the same solvent and the solvent plug used to modify the sample. That is, the subsequent plug-solvent can have the same composition as the plug-solvent. However, the subsequent plug-solvent is injected after the analytical run and typically has a volume that is on the order of 2 times or greater volume than the volume amount of solvent plug contained in the needle injector adjacent to the sample. In a preferred example, the subsequent plug-solvent volume is 10-100 times larger than the sample volume.

In an example, the mobile phase and/or the column temperature used in the methods of the present technology may be at ambient temperature (about 27 C) or above ambient temperature (e.g., 27 C to about 35 C). Mobile phase temperature may be optimized because as mobile phase temperature is brought to approach the melting temperature of self-folding, selectivity increases but so does carryover.

Anion exchange (AEX) separations (such as, purification) for oligonucleotides and nucleic acids have, in the past, limited use due to the lack of method robustness. For example, conventional methods have suffered from poor recovery and high carryover effects which are often observed with large nucleic acids. The present technology has addressed these problems. In particular, the present technology substantially decreases carryover by shortening the analyte's column residence time by starting a mobile phase gradient with comparatively high counter ion concentrations (e.g., up to 100 mM). Further, the present technology applies an injection approach where the drawn sample is bracketed or adjacent to one or more solvent plugs that modulates or reduces charged interactions and leads to improved method robustness/repeatability. The present technology also allows for the use of ion exchange chromatography for assay methods.

Solvent strength mismatch is a term used to describe a situation where the injection solvent and the mobile phase have different eluent strengths. Solvent strength mismatch is especially problematic when the sample solvent is stronger than the mobile phase composition. Such a situation often results in partial- or total breakthrough effects or at least in peak fronting or splitting. Effects like these have been frequently encountered in multi-dimensional separations and hydrophilic interaction chromatography (HILIC) analysis. To limit strong solvent effects, the sample can be introduced onto the column by applying a specially programmed injection sequence where the drawn sample is bracketed by a dilutive set of salt plug solvents. In some examples, the salt plug solvents are added before the sample plug is added; in other examples, the salt plug solvents are added after the sample plug is added.

Additionally, the present technology may optimize conditions (e.g., pH, salt concentrations, temperature, etc.) to avoid denaturation of the nucleic acid-based analytes. Without wishing to be bound by theory, the present technology demonstrates a relationship between single nucleic acid-based denaturation and carryover as well as selectivity and resolution.

The following examples illustrate the efficient solutions to significantly reduce carryover occurring in AEX separations.

### EXAMPLES

### Example 1-mRNA Sample Preparation

A sample and mobile phase were prepared as follows:

CleanCap™ EPO mRNA (5 moU; length: 858 nucleotide), luciferase (LUC) mRNA (length: 1929 nucleotide) and Cas9 mRNA (length: 4521 nucleotide) were purchased from TriLink Biotechnologies (San Diego, CA, USA). Samples were diluted to 25 μg/mL in water and injected without further preparation (for conventional methods not including the plug solvents of the present technology).

Tris-(hydroxymethyl)aminomethane (TRIS), guanidine hydrochloride (Gdn-HCl) and sodium bromide (NaBr) were purchased. TRIS buffer was prepared as 25 mM solution, and its pH was adjusted to about 7.6. This 25 mM TRIS buffer was used as mobile phase A. For mobile phase B, 2 M Gdn-HCl or 2 M NaBr was dissolved in 25 mM TRIS buffer.

For plug injections (i.e., utilizing methods of the present technology), either mobile phase B or a solution of 2 M NaBr in 10× strength BioResolve™ CX pH concentrate B (a buffer having a pH of 10.2 and commercially available from Waters Technologies Corporation part no. 186009064) were used.

TABLE 1
LC Experimental Conditions

| | |
|---|---|
| LC System: | ACQUITY ™ H-Class Bio Plus (quaternary) |
| Detection: | UV detection at 260 nm |
| Vials: | Polypropylene Vials (P/N 186002639— |
| | Waters Technologies Corporation) |
| Column: | Gen-Pak FAX Anion-Exchange Column, |
| | 2.5 μm, 4.6 mm × 100 mm (P/N WAT015490— |
| | Waters Technologies Corporation) |
| Column Temp.: | Ambient to 45° C. |
| Sample Temp.: | 5° C. |
| Injection Volume: | 2.0 μL (sample) |
| Bracketed injection | 1.0 μL (salt pre-plug) + 2.0 μL (sample) + 1.0 μL |
| sequence: | (salt post-plug) |
| Flow Rate: | 0.6 mL/min |
| Mobile Phase A: | 25 mM TRIS in water (pH = 7.6) |
| Mobile Phase B: | 2M guanidine-HCl (Gdn-HCl) in 25 mM |
| | TRIS (pH = 7.6) or |
| | 2M sodium-bromide (NaBr) HCl in 25 mM |
| | TRIS (pH = 7.6) |
| Gradient: | Recommended steep gradients for fast separation: |
| | for 2M Gdn-HCl mobile phase: 0-25% B in 6 min |
| | for 2M NaBr mobile phase: 15-50% B in 7 min |
| | Recommended shallow gradient for higher |
| | selectivity: |
| | for 2M NaBr mobile phase: 12-35% B in 15 min |

To prepare the columns for the mobile phase, the columns were conditioned by equilibrating with a minimum of 20-50 column volumes of the mobile phase to be used. Then a few consecutive (3-4) high mass load (e.g., 5-10 μg) injections of the sample of interest to condition the active sites of the stationary phase were performed.

### Example 2—The Effect of Residence Time on mRNA Carryover in AEX

Initial experiments were conducted before using the present technology to determine the effect of residence time on mRNA carryover in AEX.

These experiments suggested that the length of time an mRNA is allowed to bind to the stationary phase may correlate with carryover.

The effect of residence time on carryover has been studied experimentally in a systematic way. A Gdn-HCl gradient of 0-25% B in 6 min was programmed and various initial isocratic holding times (at 0% B) were set prior to the start of the gradient. Namely, 0, 0.5, 1, 2, 4 and 8 min initial isocratic holds were set. EPO and Cas9 mRNA samples were injected, and carryover was measured (in %) in the blank injection following the sample injection. Note that these samples were not injected with the solvent plugs of the present technology.

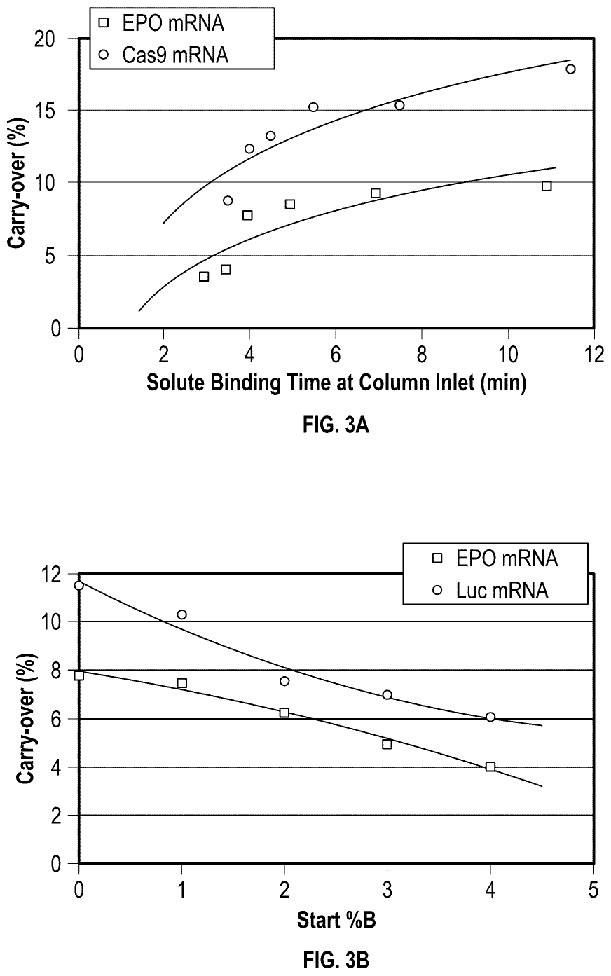

FIG. 3A shows the obtained carryover as a function of solute binding time. The plot suggests there is indeed a correlation between carryover and binding time. The shorter the binding time, the lower the carryover. As predicted, these experiments suggest that short analytical runs should be applied as a means to limit carryover. When limiting the retention time to about 3 minutes, as low as 4-8% carryover was found in contrast to 10-20% carryover observed with long gradients.

### Example 3—The Effect of Mobile Phase on mRNA Carryover in AEX

In a separate experiment before using the present technology, the impact of the initial strength of the mobile phase was measured. Without being bound by theory, the present technology surprisingly reveals that if solute spreading occurs on the surface of the stationary phase, spreading is less significant (or slower) if weaker interactions occur upon initial binding. Therefore, the gradient time was fixed, and the initial % B composition was varied as 0, 1, 2, 3 and 4% B. FIG. 3B shows the observed carryover for EPO and Luc mRNAs as a function of starting mobile phase composition. There is an obvious trend, the higher the start % B (concentration % of B in the mobile phase), the lower the carryover. Starting the gradient at 4% B (˜80 mM counterion) instead of 0% B reduced the carryover by a factor 2. This observation suggests that the gradient should start at a reasonably high % B mobile phase composition (i.e., 50-100 mM counterion) instead of 0% B.

In certain examples, the effect of binding time and of start % of a B solvent in a mobile phase gradient may be optimized based on the sample of interest.

Systematic experiments have been performed to identify the most important factors of a sequenced injection and how they affect the carryover of mRNA on an AEX separation. The following factors have been studied: (1) volume of a salt plug added before the sample, (2) volume of a salt plug added after the sample, (3) volume of the bracketing salt plugs in sum, (4) type of salt employed (e.g., NaCl, (NH₄)₂SO₄, Gdn-HCl and/or NaBr), and (5) the pH of the salt plug.

### Example 4—The Effect of Solvent Plug to Sample Volume Ratio on mRNA Carryover and Recovery

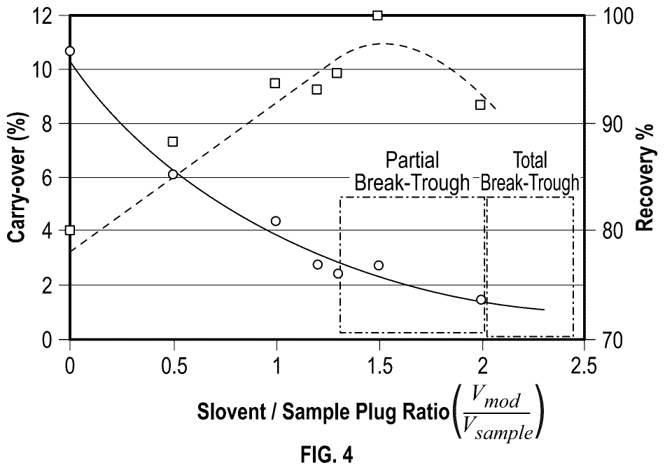

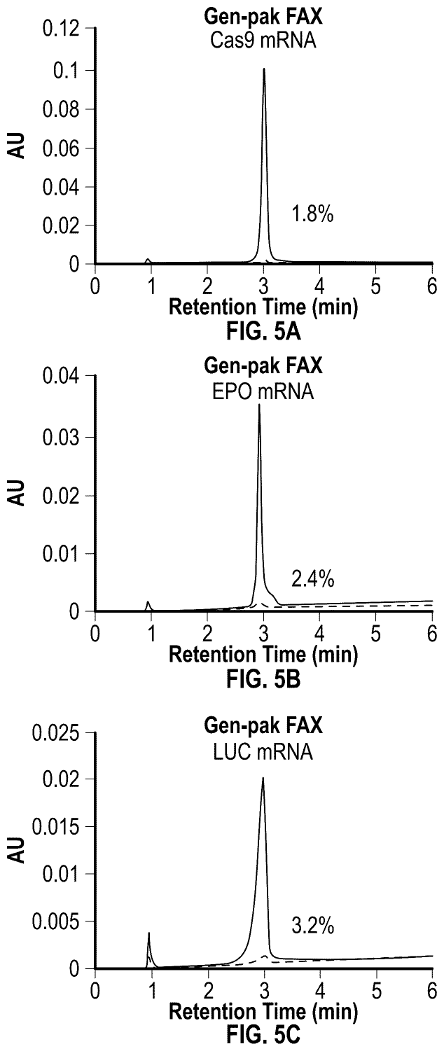

FIG. 4 shows the change in EPO mRNA's carryover and recovery as a function of modulating solvent plug to sample volume ratio

$$( \frac{{V}_{m o d}}{{V}_{sample}} ) ,$$

which can be used in a case where the volume of the pre- and post-plugs are identical. The figure shows that carryover decreases and recovery increases until

$$( \frac{{V}_{m o d}}{{V}_{sample}} )$$

reaches a value of about 1.2-1.3. Beyond this “limit” value, a fraction of the injected sample volume is taken by the strong modulating plug and a partial breakthrough peak appears on the chromatogram. If

$$( \frac{{V}_{m o d}}{{V}_{sample}} ) \geq 2$$

then the entire mRNA peak elutes at the column's dead time (total break-through). When setting a

$$( \frac{{V}_{m o d}}{{V}_{sample}} ) \approx 1 - 1.2 ,$$

as low as 2-3% carryover can be reached instead of 10-20% which is often observed without salt plug modulation.

The conditions for testing this carryover were as follows: Column: Gen-Pak FAX, commercially available from Waters Technologies Corporation 100×4.6 mm, 2.5 μm, mobile phase A: 25 mM TRIS, pH=7.6, mobile phase B: 25 mM TRIS, pH=7.6+2 M NaBr, F=0.6 mL/min, gradient: 15-50% B in 7 min, ambient temperature (˜22° C.). Sample: EPO mRNA (2 μL injected), modulator plug: mobile phase B.

The present technology is not limited in the type of AEX stationary phases and may include, both weak and strong AEX phases. In a preferred example, the AEX uses a weak exchanger. One of ordinary skill in the art would be able to adjust based on the sample and conditions. Differences may be observed between weak and strong ion exchangers, which, without being bound by theory, is likely related to differences in ligand density, ligand accessibility (morphology) and the possibility of additional interactions (e.g., H-bonding).

### Example 5—Testing Carryover and Peak Shape with Different WAX Columns

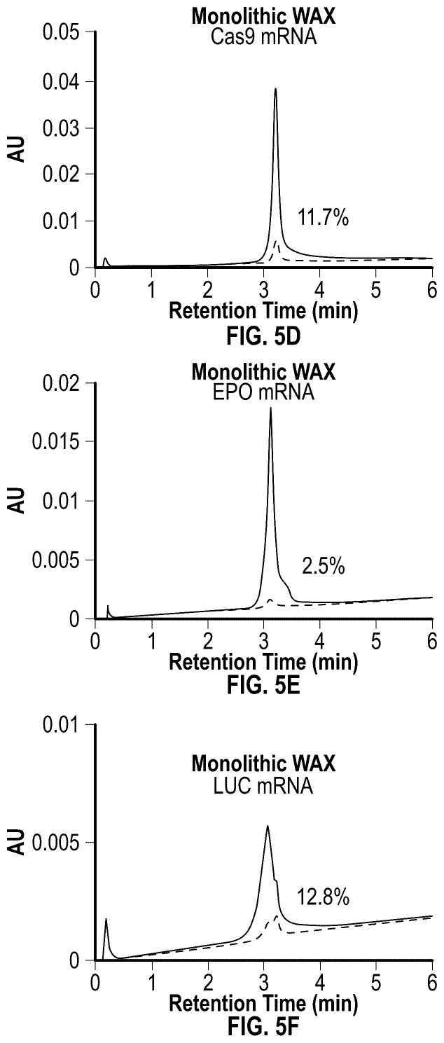

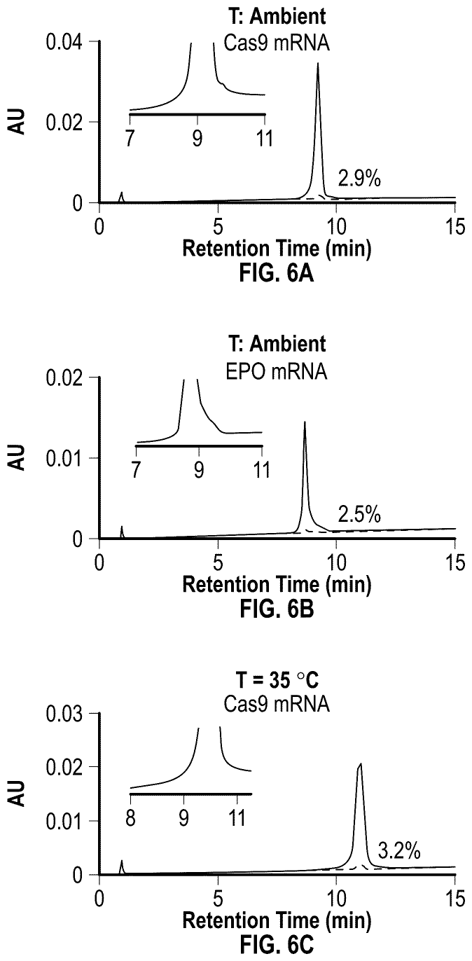

FIGS. 5A-5F show a comparison of carryover percentage and peak shape between a Gen-Pak FAX weak anion-exchange column (FIG. 5A-FIG. 5C) and a monolithic WAX column (FIG. 5D-FIG. 5F) using the same optimized bracketed injection for both columns. The Gen-Pak FAX column exhibited significantly lower carryover and comparable resolution with some instances of significantly improved peak sharpness with about 2% carryover.

The conditions were as follows: Columns: Gen-Pak FAX 100×4.6 mm, 2.5 μm (left), and monolithic WAX 4.95×5.2 mm (right). Gradient conditions: 15-50% B in 7 min, ambient temperature (˜22° C.), salt solvent plug: 2 M NaBr in pH 10.2 buffer. Bracketing injection: 1 μL modulator pre-plug+2 μL sample+1 μL modulator post-plug. The numbers expressed in % correspond to the carryover % observed in blank injection following the sample injection.

In AEX chromatography, increasing temperature may result in fewer and more defined peaks for RNA samples. Without being bound by theory, this phenomenon may be attributed to diminished secondary structure. Moreover, experimentation demonstrated an increase in retention may be observed at higher temperatures, which can also be explained by the loss of self-structure. The present technology discovered through experimentation that temperature also has significantly impacts mRNA recovery and carryover in AEX.

Changes in carryover due to temperature effects are difficult to predict, since many parameters might change the strength of the interactions between the mRNAs, the aqueous mobile phase and the stationary phase. In the presence of large amounts of salt, solvophobic effects, salting-out, salting-in, dehydration of the mRNA and structural rearrangements might all occur to some varying degrees. Therefore, the effect of temperature for bracketed injections was studied.

### Example 6—the Effect of Temperature on Peak Shape, Selectivity, and Separation Profiles

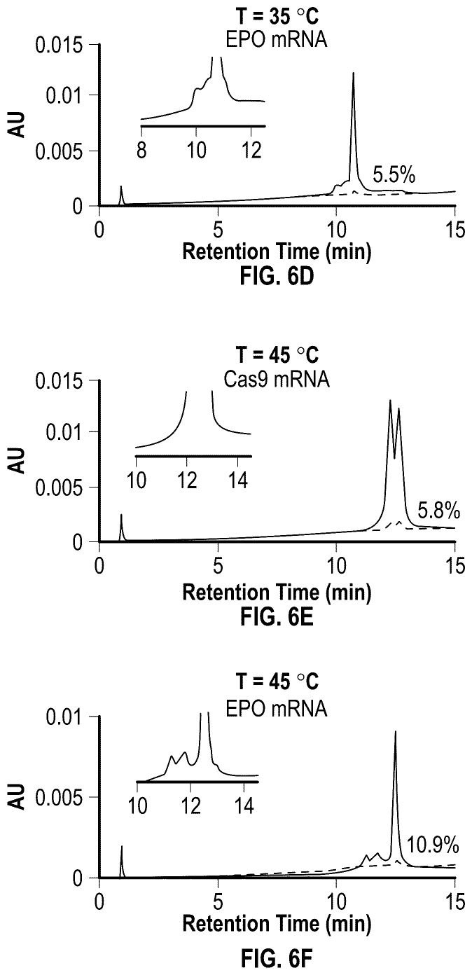

FIGS. 6A-6F show the chromatograms obtained when injecting EPO and Cas9 mRNA samples at ambient (FIG. 6A and FIG. 6B) versus elevated temperatures (T=35 (FIG. 6C and FIG. 6D) and 45° C. (FIG. 6E and FIG. 6F)). Carryover seems to increase with temperature which is in-line with observed retention increases (suggesting stronger binding at elevated temperature). As such, while temperatures are not particularly limited with the present technology, ambient temperature are preferred.

The conditions were as follows: Column: Gen-Pak FAX 100×4.6 mm, 2.5 μm, mobile phase A: 25 mM TRIS, pH=7.6, mobile phase B: 25 mM TRIS, pH=7.6+2 M NaBr, F=0.6 mL/min, gradient: 12-35% B in 15 min (shallow gradient), modulator solvent plug: mobile phase B. Bracketing injection: 1 μL modulator pre-plug+2 μL sample+1 μL modulator post-plug. Temperature: ambient (left), 35° C. (middle) and 45° C. (right).

Peak shape, selectivity and separation profiles significantly change with temperature, and there appears to be some corresponding advantageous effects on resolution of sample components. In an example, two chromatographic methods of the present technology are employed: one operated at ambient conditions and another running an elevated column temperature. The method with ambient (low) temperatures may be suitable for content/concentration determining measurements while the elevated temperature method can be valuable to investigate biophysical properties and the chemical heterogeneity of the mRNA.

### Example 7—Injection of Subsequent Salt Plug Solvent to Further Reduce Carryover

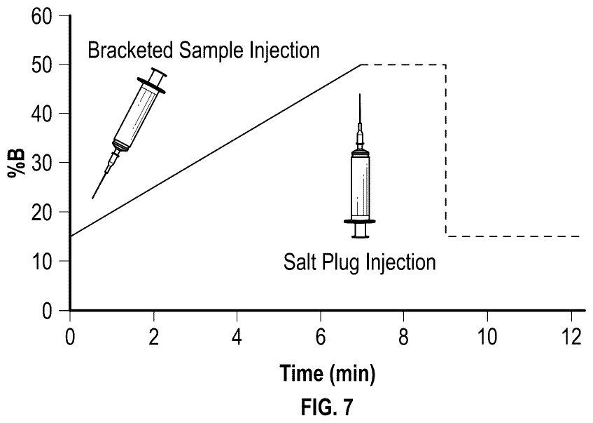

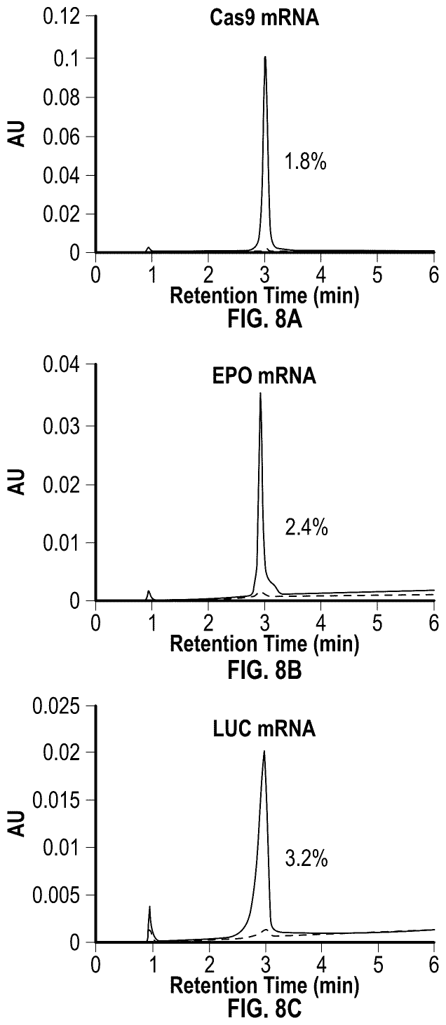

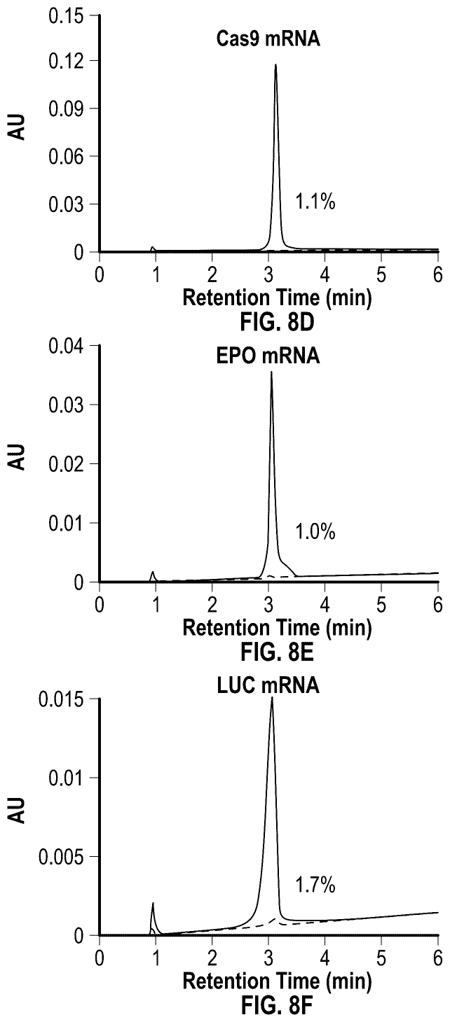

FIG. 7 shows an example in which a bracketed injection is followed by a large volume salt plug injection (such as 10-100 times larger than the sample volume; e.g., 20-200 μL on a 4.6 mm ID column) after a time interval. Carryover is further reduced as demonstrated in FIGS. 8A-8F. As shown in FIGS. 8A-8C, with bracketed injection carryover is reduced to about 2%. As shown in FIGS. 8D-8F, with a subsequent salt plug injection, carryover was as low as about 1%.

### Example 8—Breakthrough Based on the Volumetric Solvent/Sample Plug Ratio and pH

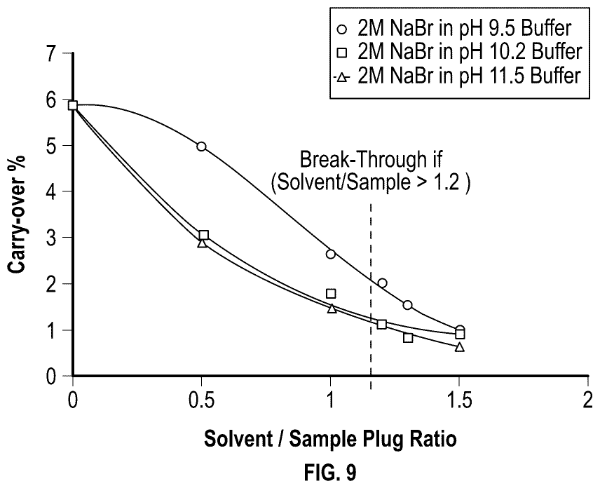

FIG. 9 shows the carryover effect based on the volumetric solvent/sample plug ratio with varying pH while using the same salt concentration of 2M NaBr. As can be observed, breakthrough occurs above a solvent/sample plug ratio of 1.2.

### Example 9—Peak Analysis Based on Salt Plug Solvent pH

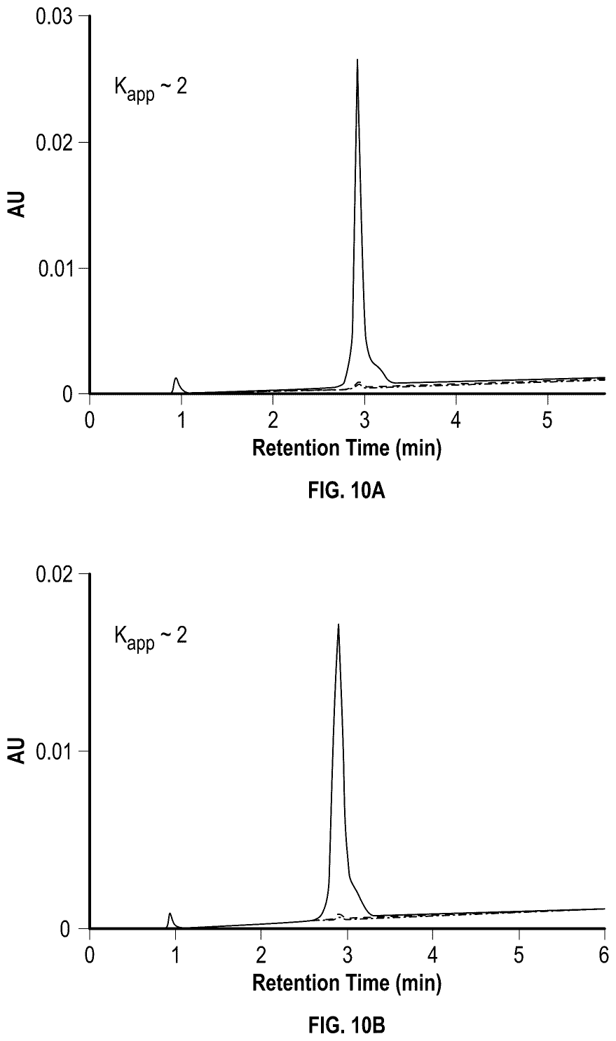

FIGS. 10A and 10B illustrates a comparison of the effect of salt plug solvent pH (10.2 vs 11.5) on peak analysis. As can be seen, a pH of 10.2 demonstrated a sharper peak. In FIG. 10A, the sample solvent plugs are 1+1 μL 2 M NaBr in BioResolve™ CX-B 10×pH=10.2; sample: 2 μL. In FIG. 10B, the sample solvent plugs are 1+1 μL 2 M NaBr in in 25 mM Na₂CO₃ pH=11.5; sample: 2 μL.

### CONCLUSION

The above experiments reveal that the present technology reduces carryover and thus improves method robustness. Of the ion-exchange columns tested, Gen-Pak FAX column (i.e., an ion-exchange column that contains a weak anion exchanger comprising a diethylaminoethyl-functionalized non-porous resin) has been confirmed to give some of the most effective separations of large, single stranded nucleic acid samples.

The experiments also showed that a short analysis time (less than 4 minutes) is favorable in terms of carryover. In preferred examples, the methods of the present technology would start the gradient with a relatively high eluent strength.

Finally, the results demonstrate that injection in which the sample is bracketed or adjected to salt solvent plugs that modulate sample charge. These plugs contain high concentrations of salt and are buffered to have a high enough pH without denaturing the analyte of interest (e.g., about 10). This injection helps reduce the strength of solute binding at the head of the column, thus improving recovery and carryover. The volume and the ratio of the modulator plug needs may be optimized for each individual method setup. With the parameters used in the above examples, a ratio for

$$( \frac{{V}_{m o d}}{{V}_{sample}} )$$

of approximately 1 proved to be optimally effective. By using a bracketed injection mode with a Gen-Pak FAX column, the present technology was able to reduce the carryover of large mRNAs to ˜2%, in contrast to the 10-20% carryover which has often observed with conventional AEX methods.

While this disclosure has been particularly shown and described with reference to examples embodiments thereof, it will be understood by those skilled in the art that various changes in form and details may be made therein without departing from the scope of the technology encompassed by the appended claims.

What is claimed is:

1. A method of ion-exchange chromatography comprising the steps of:
loading a bracketed sample into an injector needle apparatus by:
(a) adding a first-plug solvent comprising a first salt, and wherein the first-plug solvent has a basic pH;
(b) adding a sample plug;
(c) adding a second-plug solvent comprising a second salt, and wherein the second-plug solvent has a basic pH; and
injecting the bracketed sample into an ion-exchange column for chromatographic separation.

2. The method of claim 1, further comprising the step of injecting into the ion-exchange column a subsequent salt plug injection subsequent to injecting the bracketed sample.

3. The method of claim 2, wherein the subsequent salt plug injection is greater in volume than the first-plug solvent or the second-plug solvent.

4. The method of claim 1, wherein the ion-exchange column contains a weak anion exchanger comprising a diethylaminoethyl-functionalized non-porous resin.

5. The method of claim 1, wherein the first-plug solvent and the second-plug solvent comprise the same composition.

6. The method of claim 1, wherein the sample comprises a nucleotide, oligonucleotide, nucleic acid, or fragments or derivatives thereof.

7. The method of claim 1, wherein the first-plug solvent and/or the second-plug solvent comprises sodium bromide or guanidine HCl.

8. The method of claim 1, wherein the first salt concentration of the first-plug solvent and/or the second salt concentration of the second-plug solvent is greater than 1M of sodium bromide.

9. The method of claim 8, wherein the first salt concentration of the first-plug solvent and/or the second salt concentration of the second-plug solvent is 2M of sodium bromide.

10. The method of claim 1, wherein the volumetric ratio of first-plug solvent to second-plug solvent in the injector needle apparatus is 1:1.

11. The method of claim 1, wherein the pH in the first-plug solvent and/or the second-plug solvent is about 10.

12. The method of claim 1, wherein each of the first-plug solvent and second-plug solvent further comprises a buffer.

13. The method of claim 1, wherein a solvent plug volume is at least equal to a sample volume; wherein the solvent plug volume consists of a volume filled by first-plug solvent and the second-plug solvent.

14. The method of claim 13, wherein the solvent plug volume is 1.2 times the sample plug volume.

15. The method of claim 1, wherein the bracketed sample is injected into the ion-exchange column using a mobile phase gradient.

16. The method of claim 15, wherein the mobile phase gradient comprises a first solvent and a second solvent and excludes 0% of the second solvent.

17. The method of claim 1, wherein the ion-exchange column is run at about ambient temperature or above ambient temperature.

18. A method of ion-exchange chromatography comprising the steps of:
loading a sample into an injector needle apparatus to form a modified sample by:
(a) adding a salt plug solvent having a basic pH and comprising 2M NaBr and a buffer; and
(b) adding the sample; and
injecting the modified sample into an ion-exchange column for chromatographic separation.

19. The method of claim 18, wherein the salt plug solvent is added prior to adding the sample.

20. The method of claim 18, wherein the salt plug solvent is added after adding the sample.
